(19) United States
(12) Patent Application Publication
(10) Pub. No.: US 2010/0256350 A1
(43) Pub. Date: Oct. 7, 2010

(54) MICROFLUIDIC APPARATUS FOR SEPARATING TARGET SUBSTANCE AND METHOD OF PURIFYING THE TARGET SUBSTANCE FROM SAMPLE

(75) Inventors: Joo-won RHEE, Seoul, (KR); Chin-sung PARK, Yongin-si, (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si, (KR)

(21) Appl. No.: 12/630,126

(22) Filed: Dec. 3, 2009

(30) Foreign Application Priority Data
Apr. 6, 2009 (KR) 10-2009-0029489

(51) Int. Cl. C07H 21/00; B01D 33/00

(52) U.S. Cl. 536/25.4; 210/196

(57) ABSTRACT

A microfluidic apparatus includes a filter unit including an entrance, a separation portion in which eluant contacts microparticles to separate a target substance bound to surfaces of the microparticles from the microparticles, an exit, and a filter blocking leakage of the microparticles through the exit, a fluid supply portion selectively supplying the eluant and a sample solution including the microparticles capturing the target substance, to the filter unit, and a resupply unit supplying the eluant passed through the filter unit back to the filter unit.

### CROSS-REFERENCE TO RELATED APPLICATIONS

This application claims priority to Korean Patent Application No. 10-2009-0029489, filed on Apr. 6, 2009, and all the benefits accruing therefrom under 35 U.S.C. §119, the disclosures of which are incorporated herein in their entirety by reference.

### BACKGROUND

1. Field

One or more embodiments of the invention relate to a microfluidic apparatus for separating a target substance, such as nucleic acid, and protein from a sample using microparticles, and a method of purifying the target substance from the sample.

2. Description of the Related Art

Samples related to clinic or environment are analyzed by a series of biochemical, chemical, and mechanical processes. Technologies for diagnosis or monitoring of biochemical samples have been actively developed. A molecule diagnosis method based on nucleic acid, which exhibits superior accuracy and sensitivity, is increasingly used for diagnosis of infectious diseases or cancer, or pharmacogenomics.

The nucleic acid used for a polymerase chain reaction (“PCR”) apparatus, a molecular diagnostic apparatus, a point of care testing (“POCT”) apparatus, a nucleic acid diagnostic apparatus, or a nucleic sequence diagnostic apparatus is purified by being separated from a biochemical sample. The purification of the nucleic acid is carried out by capturing nucleic acid only from the biochemical sample using a probe that is specifically coupled to the nucleic acid, and then separating the captured nucleic acid from the probe by using eluant.

To improve nucleic acid purification efficiency, a large amount of eluant is used. In this case, the concentration of nucleic acid of the collected eluant is very low. In contrast, a small amount of eluant is used to increase the concentration of nucleic acid of the collected eluant. In this case, the purification efficiency is lowered.

### SUMMARY

One or more embodiments of the invention include a microfluidic apparatus for capturing a target substance from a sample using microparticles, and separating the microparticles and a fluid including the captured target substance, and a method of purifying the target substance from the sample.

One or more embodiments of the invention include a microfluidic apparatus which may improve a purification efficiency by using a limited amount of eluant, and a method of purifying the captured target substance from the sample.

One or more embodiments of the invention include a microfluidic apparatus which may employ a filter unit suitable for the purpose of use.

Additional aspects will be set forth in part in the description which follows and, in part, will be apparent from the description, or may be learned by practice of the illustrated embodiments.

According to one or more embodiments of the invention, a microfluidic apparatus includes a filter unit including an entrance, a separation portion in which eluant contacts microparticles to separate a target substance bound to surfaces of the microparticles from the microparticles, an exit, and a filter blocking leakage of the microparticle through the exit, a fluid supply portion selectively supplying to the filter unit the eluant and a sample solution including the microparticles including the surfaces to which the target substance is bound, and a resupply unit supplying the eluant passed through the filter unit back to the filter unit.

The resupply unit may include a resupply path connecting the exit and the entrance of the filter unit, and a resupply pump transferring the eluant to the entrance of the filter unit along the resupply path.

The fluid supply portion may include an eluant chamber accommodating the eluant. The resupply unit may include a resupply path connecting the exit and the entrance of the filter unit to each other, and a resupply pump transferring the eluant to the entrance of the filter unit along the resupply path.

The microfluidic apparatus may further include a fluid accommodation portion accommodating the eluant exhausted through the exit of the filter unit. The fluid accommodation portion may include a target substance accommodation chamber accommodating the eluant including the target substance separated from the microparticles, and an accommodation valve selectively connecting the target substance accommodation chamber and the exit of the filter unit. The resupply unit may include a resupply path connecting the target substance accommodation chamber and the entrance, and a resupply pump transferring the eluant to the entrance along the resupply path. The fluid supply portion may include an eluant chamber accommodating the eluant The resupply unit includes a resupply path connecting the target substance accommodation chamber and the eluant chamber, and a resupply pump transferring the eluant to the entrance along the resupply path.

The resupply pump may be located on the resupply path.

The microfluidic apparatus may further include a platform on which the fluid supply portion, the fluid accommodation portion, and the resupply unit are provided. The filter unit is separated from the platform, and the entrance and the exit are connected to the fluid supply portion, the fluid accommodation portion, and the resupply unit via first and second connection members.

According to one or more embodiments of the invention, a method of purifying a target substance includes packing microparticles including surfaces to which the target substance is bound, in a filter unit, separating the target substance from the microparticles by supplying eluant to the filter unit, and resupplying the eluant including the target substance exhausted from the filter unit at least one time to the filter unit.

### BRIEF DESCRIPTION OF THE DRAWINGS

These and/or other aspects will become apparent and more readily appreciated from the following description of the embodiments, taken in conjunction with the accompanying drawings of which:

FIG. 1 schematically illustrates an exemplary embodiment of a structure of a microfluidic apparatus according to the invention;

FIG. 2 illustrates an exemplary embodiment of a filter unit employed in the microfluidic apparatus of FIG. 1;

FIG. 3A illustrates an exemplary embodiment of a closed state of a supply valve and an accommodation valve;

FIG. 3B illustrates an exemplary embodiment of an open state of the supply valve and the accommodation valve;

FIG. 4 schematically illustrates an exemplary embodiment of a structure of a resupply pump according to the invention;

FIG. 5 schematically illustrates another exemplary embodiment of a structure of a microfluidic apparatus according to the invention;

FIG. 6 schematically illustrates another exemplary embodiment of a structure of a microfluidic apparatus according to the invention;

FIG. 7 schematically illustrates another exemplary embodiment of a structure of a microfluidic apparatus according to the invention; and

FIG. 8 schematically illustrates another exemplary embodiment of a structure of a microfluidic apparatus according to the invention.

### DETAILED DESCRIPTION

Reference will now be made in detail to embodiments, examples of which are illustrated in the accompanying drawings, wherein like reference numerals refer to the like elements throughout. In this regard, the illustrated embodiments may have different forms and should not be construed as being limited to the descriptions set forth herein. Accordingly, the embodiments are merely described below, by referring to the figures, to explain features of the description. In the drawings, the size and relative sizes of layers and regions may be exaggerated for clarity.

It will be understood that when an element or layer is referred to as being “on”, “connected to” or “coupled to” another element or layer, the element or layer can be directly on, connected or coupled to another element or layer or intervening elements or layers. In contrast, when an element is referred to as being “directly on,” “directly connected to” or “directly coupled to” another element or layer, there are no intervening elements or layers present. Like numbers refer to like elements throughout. As used herein, the term “and/or” includes any and all combinations of one or more of the associated listed items.

It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the invention.

Spatially relative terms, such as “lower”, “upper” and the like, may be used herein for ease of description to describe the relationship of one element or feature to another element(s) or feature(s) as illustrated in the figures. It will be understood that the spatially relative terms are intended to encompass different orientations of the device in use or operation, in addition to the orientation depicted in the figures. For example, if the device in the figures is turned over, elements described as “lower” relative to other elements or features would then be oriented “upper” relative to the other elements or features. Thus, the exemplary term “lower” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

Hereinafter, the invention will be described in detail with reference to the accompanying drawings.

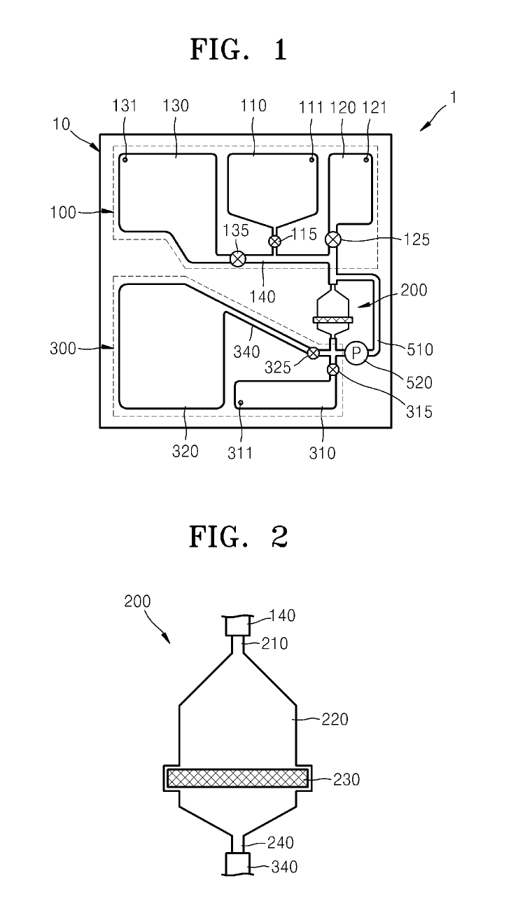

FIG. 1 schematically illustrates an exemplary embodiment of a structure of a microfluidic apparatus 1 according to the invention. Referring to FIG. 1, the microfluidic apparatus 1 according to the illustrated embodiment may include a platform 10 in which a plurality of chambers, a plurality of channels connecting the chambers, and a plurality of valves regulating the flow of a fluid through the channels are provided.

The platform 10 may be collectively formed by two or more separate and individual plates coupled to each other. In one exemplary embodiment, the platform 10 may be formed by coupling an upper plate to a lower plate in which the above-described plurality of chambers, plurality of channels connecting the chambers, and plurality of valves regulating the flow of a fluid through the channels for the flow of a fluid are disposed, such as by engraving. Alternatively, the platform 10 may be formed by inserting a sectioning plate for defining the plurality of chambers, plurality of channels connecting the chambers, and plurality of valves regulating the flow of a fluid through the channels for the flow of a fluid between upper and lower plates. The platform 10 is not limited to the above shapes and may be manufactured in a variety of shapes.

The microfluidic apparatus 1 captures a target substance included in a sample solution, for example, a biochemical sample, using microparticles. The microfluidic apparatus 1 separates the target substance from the microparticles, thereby obtaining a purified fluid including the target substance from the biochemical sample. A probe having a characteristic of being specifically coupled to the target substance is provided on surfaces of the microparticles.

In one exemplary embodiment, silica particles or inorganic oxides may be employed as the microparticles. The inorganic oxides may be, for example, glass particles, alumina, zirconia, or titania. The biological sample may be, for example, cell suspension including a microorganism, human blood, urine, or saliva, but not limited thereto. Also, the target substance is not specially limited and may be, for example, nucleic acid, protein, peptide, an antibody, or hormone. The nucleic acid may be deoxyribonucleic acid (“DNA”) or ribonucleic acid (“RNA”).

Referring to FIG. 1, the microfluidic apparatus 1 may include a fluid supply portion 100 and a filter unit 200. The fluid supply portion 100 may include a sample chamber 110 containing a sample solution (e.g., biological sample) including a target substance, and an eluant chamber 120 containing eluant for separating the target substance from microparticles.

In an exemplary embodiment, the microparticles may be contained in the sample chamber 110 with the sample solution. Although it is not illustrated, a microparticle accommodation chamber may be separately provided to accommodate a fluid including the microparticles. In this case, the microparticle accommodation chamber is connected to the sample chamber 110 via a channel (not shown). When a purification process is performed, the fluid including the microparticles is supplied to the sample chamber 110 and mixed with the sample solution. To improve the purity of the target substance to be purified, the fluid supply portion 100 may further include a washing solution chamber 130 containing a washing solution.

The filter unit 200 filters the microparticles from the fluid including both the microparticles and the target substance. FIG. 2 illustrates the filter unit 200 employed in the microfluidic apparatus 1 of FIG. 1. In the illustrated exemplary embodiment, the filter unit 200 is a sieve type filter unit.

Referring to FIG. 2, the filter unit 200 includes an entrance 210, a separation portion 220 in which the microparticles and the target substance are separated from each other as the eluant and the microparticles are contacted with each other, an exit 240, and a filter 230 located between the exit 240 and the separation portion 220. The filter 230 may be a member including a porous material that blocks the microparticles and passes the fluid including the target substance only. The filter 230 may be appropriately selected in consideration of the diameter of each microparticle in use.

The microfluidic apparatus 1 may further include the fluid accommodation portion 300. The fluid accommodation portion 300 may include a target substance accommodation chamber 310, and a waste chamber 320. The target substance accommodation chamber 310 accommodates the eluant including the target substance originally in the sample solution and separated from the microparticles after processing through the filter unit 200. The waste chamber 320 accommodates a remainder of the sample solution (e.g., without the target substance) and the washing solution passing through the filter unit 200. The target substance accommodation chamber 310 may includes an exhaust hole 311 to drain the eluant including the captured target substance.

The sample chamber 110, the eluant chamber 120, and the washing solution chamber 130 are connected to the entrance 210 of the filter unit 200 via a supply channel 140. The supply channel 140 may be a single unitary and continuous channel fluidly connected to each of the sample chamber 110, the eluant chamber 120, and the washing solution chamber 130. Valves 115, 125, and 135 are supply valves that selectively connect and disconnect the sample chamber 110, the eluant chamber 120, and the washing solution chamber 130 to and from the supply channel 140, respectively. By operating the valves 115, 125, and 135, the sample solution including the target substance, the eluant, and the washing solution may be selectively supplied to the filter unit 200. The target substance accommodation chamber 310 and the waste chamber 320 are connected to the exit 240 of the filter unit 200 via an exhaust channel 340.

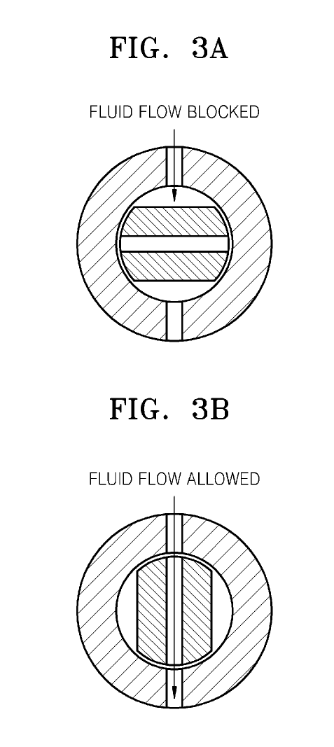

Valves 315 and 325 are accommodation valves to selectively connect and disconnect the target substance accommodation chamber 310 and the waste chamber 320 to and from the exhaust channel 340. The valves 115, 125, 135, 315, and 325 may have any shape or configuration capable of opening and closing, such as to allow passage of fluid or restrict passage of fluid, respectively. In one exemplary embodiment, a two-way valve having two states, such as of blocking the flow of a fluid as shown in FIG. 3A, and allowing the flow of a fluid as shown in FIG. 3B, by a pneumatic apparatus (not shown) or a manual operation, may be employed.

The fluid supply portion 100 is not limited to the above-described structure. In one exemplary embodiment, the fluid supply portion 100 may have a shape of a channel (not shown) connected to the entrance 210 of the filter unit 200. The shape of a channel includes configurations such as a tubular or groove-like passage way, such as indicated by the supply channel 140 in FIG. 1. Instead of each of the sample chamber 110, the eluant chamber 120, and the washing solution chamber 130 being connected to the common supply channel 140, the sample chamber 110, the eluant chamber 120, and the washing solution chamber 130 may themselves form a channel finally connected to the entrance 210 of the filter unit 200. In this case, the sample solution, the washing solution, and the eluant may be sequentially supplied to the filter unit 200 via the channel.

In an alternative exemplary embodiment, the fluid accommodation portion 300 may not include the target substance accommodation chamber 310. The exit 240 of the filter unit 200 and the exhaust hole 311 are directly connected to each other by a channel (not shown), since the target substance accommodation chamber 310 is omitted. Thus, when the valve 315 is open after the separation of the target substance from the from the microparticles is completed in the filter unit 200, the elution including the target substance is exhausted to an outside of the fluid accommodation portion 300 and/or of the entire microfluidic apparatus 1 through the exhaust hole 311.

The flow of a fluid in the microfluidic apparatus 1 may be generated by, for example, an external gas pressure. To this end, gas input holes 111, 121, and 131 may be provided in the sample chamber 110, the eluant chamber 120, and the washing solution chamber 130, respectively.

In an exemplary embodiment of a method of separating a target substance using microparticles, while an efficiency of binding DNA to the surfaces of the microparticles is very high, an efficiency of eluting the target substance from the microparticles is low, thus lowering an overall separation efficiency. To address this matter, a method of allowing the eluant to pass the filter unit 200 multiple times may be taken into consideration. The microfluidic apparatus 1 may include a resupply unit for resupplying the eluant to the filter unit 200.

Referring to FIG. 1, the resupply unit includes a resupply path 510 and a resupply pump 520. In the illustrated embodiment, the resupply path 510 may be connected at a first end to the exit 240 of the filter unit 200, and may be connected at a second end opposite to the first end to the entrance 210 of the filter unit 200. The fluid supply portion 100 and the fluid accommodation portion 300 are completely outside of a flow of the resupply path 510, where only the filter unit 200 is included in the resupply path 510. A backflow prevention valve (not shown) to prevent backflow of the eluant may be provided at a connection portion between the resupply path 510 and the entrance 210 of the filter unit 200.

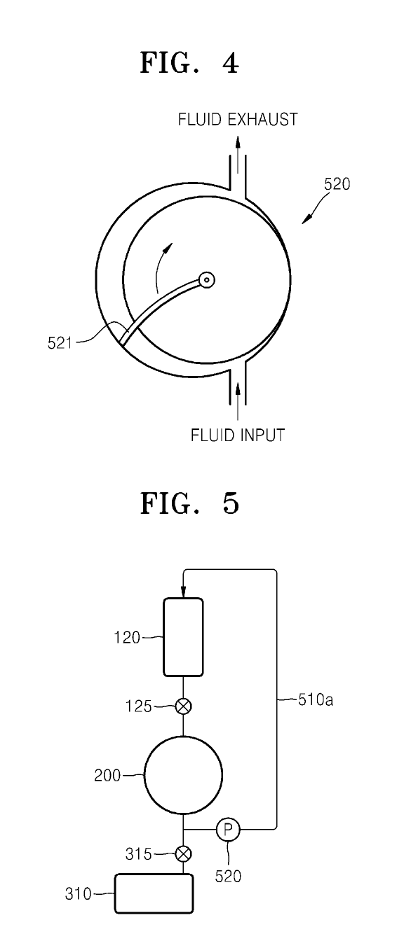

The resupply pump 520 provides a driving force to supply the eluant which has passed through the filter unit 200, back to a starting portion of the filter unit 200. The structure and type of the resupply pump 520 are not limited to the above-described ones. In one exemplary embodiment, as illustrated in FIG. 4, a vein pump including an eccentric blade 521 that is rotated (shown by the arrow in FIG. 4) may be employed as the resupply pump 520. A pneumatic apparatus may be used to rotate the eccentric blade 521. Alternatively, a pump including a microscale on-chip valves (“MOV”) structure including a polydimethylsiloxane membrane (“PDMS”) driven by pneumatic pressure may be employed as the resupply pump 520. Also, the fluid in the resupply path 510 may be moved by a gas pressure that is supplied into the resupply path 510.

In an exemplary embodiment of a process of purifying a target substance in the microfluidic apparatus 1 configured as above, the sample solution in which the target substance is originally accommodated, an eluant, and a washing solution are loaded in the sample chamber 110, the eluant chamber 120, and the cleaning chamber 130, respectively. The microparticles may be mixed in the sample solution before, at substantially the same time as, or after the sample solution including the target substance is loaded into the sample chamber 110.

In one exemplary embodiment as described above, when the microfluidic apparatus 1 is provided with the microparticle accommodation chamber accommodating a fluid including the microparticles, a gas pressure is applied to the microparticle accommodation chamber so that the fluid including the microparticles may be supplied to the sample chamber 110. In the sample chamber 110, the target substance may be bound to the surfaces of the microparticles due to the specific coupling to the probes provided on the surfaces of the microparticles.

After the sample solution including target substance and the microparticles are disposed in the sample chamber 110, the sample solution is supplied to the filter unit 200. When the valves 115 and 325 are open, and a gas pressure is applied to the sample chamber 110 via the gas input hole 111, the sample solution is supplied to the filter unit 200 via the supply channel 140. Since the microparticles are not able to pass through the filer 230, only the sample solution passes through the filter 230. The sample solution (excluding the target substance) is exhausted to the waste chamber 320 via the exhaust channel 340 since the valve 325 is open. The microparticles capturing the target substance remain in the separation portion 220 of the filter unit 200. When the exhaustion of the sample solution (excluding the target substance) to the waste chamber 320 is completed, the valve 115 is closed.

After the microparticles including the captured target substance are disposed in the separation portion 220 of the filter unit 200, the process of removing impurities adhering to and mixed with the microparticles in the separation portion 220 of the filter unit 200 is initiated, such as by opening the valve 135. The washing solution is supplied to the filter unit 200, such as by applying a gas pressure to the washing solution chamber 130 via the gas input hole 131. The impurities mixed with the microparticles in the separation portion 220, together with the washing solution, pass through the filter 230 and the exhaust channel 340, and are exhausted to the waste chamber 320 since the value 325 is still open. When the exhaustion of the impurities and the washing solution is completed, the valves 135 and 325 are closed.

After the exhaustion of the impurities and the washing solution is completed, the process of separating the target substance bound to the surfaces of the microparticles is initiated by opening the valve 125. The eluant is supplied to the filter unit 200, such as by applying a gas pressure to the eluant chamber 120 via the gas input hole 121. The specific coupling between the target substance and the probe provided on the surfaces of the microparticles is disconnected by the operation of the eluant supplied into the separation portion 220. The eluant including the separated target substance is exhausted through the exit 240 of the filter unit 200.

In an exemplary embodiment, since the valves 315 and 325 are closed, operation of the resupply pump 520 starts, the eluant including the separated target substance is supplied back to the filter unit 200 via the resupply path 510. After the eluant repeatedly passes through the filter unit 200, the valve 315 is open and the eluant including the target substance is accommodated in the target substance accommodation chamber 310.

According to the above processes, the target substance may be separated from the sample solution and then purified. Since the eluant is used to disconnect the specific coupling between the target substance and the probes of the microparticles, if the eluant sufficiently soaks the microparticles in the filter unit 200, by the recirculation of the eluant including the separated target substance through the filter unit 200, the efficiency of separation of the microparticles and the target substance may be improved.

According to the illustrated embodiment, the eluant passing through the filter unit 200 is resupplied to the filter unit 200 to increase a contact possibility between the microparticles and the eluant, so that the separation of the microparticles and the target substance may be achieved at a high efficiency. Thus, eluant including the target substance of a high concentration may be obtained by using a small amount of eluant.

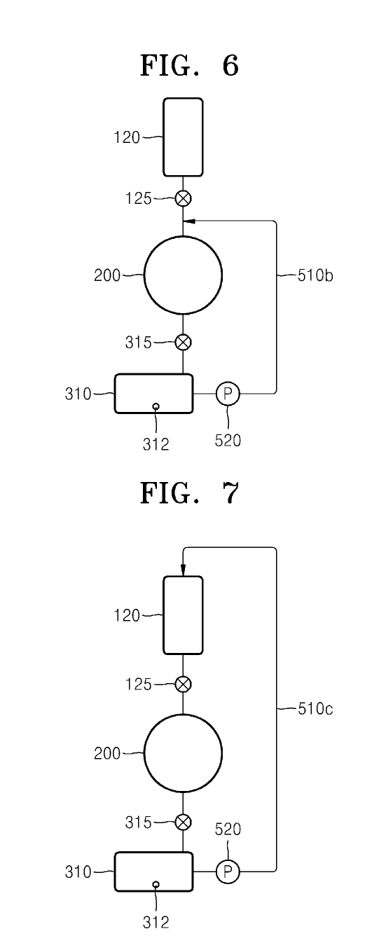

FIG. 5 schematically illustrates another exemplary embodiment of a structure of a microfluidic apparatus according to the invention. Referring to FIG. 5, a resupply path 510a connects the exit 240 of the filter unit 200 and the elution chamber 120. The target substance accommodation chamber 310 is completely outside of a recirculation path including the resupply path 510a, while the eluant chamber 120 is a portion of the recirculation path. The recirculation path includes only the resupply path 510a, the resupply pump 520 and the eluant chamber 120. The microfluidic apparatus according to the illustrated embodiment is substantially the same as that of FIG. 1, except that the eluant exhausted from the filter unit 200 is supplied to the eluant chamber 120 along the resupply path 510a and resupplied to the filter unit 200. In contrast, the microfluidic apparatus 1 in FIG. 1 includes the eluant exhausted from the filter unit 200 is supplied back to the filter unit 200 (e.g., the entrance 210).

FIG. 6 schematically illustrates another exemplary embodiment of a structure of a microfluidic apparatus according to the invention. Referring to FIG. 6, a resupply path 510b connects the target substance accommodation chamber 310 and the entrance 210 of filter unit 200. The target substance accommodation chamber 310 is a portion of a recirculation path including the resupply path 510b, while the eluant chamber 120 is completely outside of the recirculation path. The recirculation path includes only the resupply path 510a, the resupply pump 520 and the target substance accommodation chamber 310. The microfluidic apparatus according to the illustrated embodiment is substantially the same as that of FIG. 1, except that the eluant exhausted from the filter unit 200 is first accommodated in the target substance accommodation chamber 310 and then resupplied to the filter unit 200 along the resupply path 510b. In contrast, the microfluidic apparatus 1 in FIG. 1 includes the eluant exhausted from the filter unit 200 is supplied directly back to the filter unit 200 (e.g., the entrance 210). In an alternative embodiment, instead of the resupply pump 520, a gas inlet 312 through which gas pressure to resupply the eluant is provided, may be disposed in the target substance accommodation chamber 310 and the resupply pump 520 may be omitted.

FIG. 7 schematically illustrates another exemplary embodiment of a structure of a microfluidic apparatus according to the invention. Referring to FIG. 7, a resupply path 510c connects the target substance accommodation chamber 310 and the eluant chamber 120. Both the target substance accommodation chamber 310 and the eluant chamber 120 are a portion of a recirculation path including the resupply path 510c. The recirculation path includes only the resupply path 510a, the resupply pump 520, the eluant chamber 120 and the target substance accommodation chamber 310. The microfluidic apparatus according to the illustrated embodiment is substantially the same as that of FIG. 6, except that the eluant exhausted from the filter unit 200 is firstly accommodated in the target substance accommodation chamber 310, supplied to the eluant chamber 120 along the resupply path 510c, and finally resupplied to the filter unit 200. The valve 125 is closed during which the eluant is supplied from the target substance accommodation chamber 310 to the eluant chamber 120. When the supply of the eluant to the eluant chamber 120 is completed, the valve 125 is open to resupply the eluant to the filter unit 200.

An experiment of purifying DNA from a sample solution having a relatively high DNA concentration, and a sample solution having a relatively low DNA concentration is carried out using the above-described microfluidic apparatus 1.

<Specification of Filter Unit in Use>

Filter: a nitro-cellulose material having an about 1.2 micrometers (μm) pore size, manufactured by Millipore (Cat. No. RAWP04700)

Volume of a separation portion: about 30 microliters (μL)

Entrance: volume of about 0.135 μL, diameter of about 1/16 inch, length of about 0.5 millimeter (mm)

Exit: volume of about 0.135 μL, diameter of about 1/16 inch, length of about 0.5 mm

<Sample Solution>

A sample solution of 500 μL including DNA 260 microgram (μg) having a distribution of an average 1-2 kb (kilo basepair) size and impurities such as protein, salt, deoxy-nucleotide-tri phosphate (dNTP), or detergent.

<Negative Group>

Ampure polymerase chain reaction (“PCR”) clean up KIT (Cat. No. A29152) manufactured by Agencourt

<Experiments Method Using Negative Group And Result Thereof>

Agencourt SPRIstand™-Magnetic 6-tube stand is used according to Ampure KIT manual. The amount of a solution including microparticles is about 1000 μL that is twice the amount of the sample solution. DNA is captured using the microparticles and washed using 70% ethanol of about 500 μL. The microparticles are dried in the air for about 20 minutes and eluated using deionized water of about 45 μL. Since the resupply unit is not used in this illustrated experiment unlike in the microfluidic apparatus 1 of the invention, the elution is performed one time.

In the case of the negative group, since the sample solution of about 1000 μL including the microparticles is used, when the microparticles are sufficiently dried, soaked in deionized water of about 45 μL, and let stand on a magnetic stand, DNA that may be sampled using a pipette hardly exists. This is because the amount of the microparticles is too large and the amount of the deionized water used as the eluant is too small so that most deionized water is absorbed by the microparticles.

<Experiment Method Using Microfluidic Apparatus of the Illustrated Embodiment of the Invention and Result Thereof>

A microparticle solution of about 1000 μL, that is the same as one used for the negative group, is mixed with a sample solution of about 500 μL. The mixed solution passes through a filter unit for about 20 minutes at a pressure of about 20 pounds per square inch (“psi”). 70% Ethanol of about 500 μL passes through a filter unit for about 20 minutes at a pressure of about 20 psi to wash the microparticles. Then, the microparticles are sufficiently dried and eluated using deionized water of about 45 μL. An experiment of repeating the above-descried process for three cycles is performed three times.

The concentration of DNA in nanograms per microliter (ng/μL), a protein contamination level, a salt contamination level, yield, and the volume of eluant in the final eluant are obtained as follows.

[Experiment 1]

| | | DNA | Protein | Salt |
|---|---|---|---|---|
| | Concentration of | Yield | Contamination | Contamination |
| | DNA (ng/μL ) | (%) | Level (260/280) | Level (260/230) |
| Cycle 1 | 3.6 | 62 | 1.85 | 2.37 |
| Cycle 2 | 4.2 | 72 | 1.86 | 2.36 |
| Cycle 3 | 4.3 | 74 | 1.86 | 2.35 |

[Experiment 2]

| | | DNA | Protein | Salt |
|---|---|---|---|---|
| | Concentration of | Yield | Contamination | Contamination |
| | DNA (ng/μL) | (%) | Level (260/280) | Level (260/230) |
| Cycle 1 | 3.1 | 53 | 1.86 | 2.36 |
| Cycle 2 | 3.2 | 55 | 1.87 | 2.36 |
| Cycle 3 | 4.5 | 77 | 1.86 | 2.35 |

[Experiment 3]

| | | DNA | Protein | Salt |
|---|---|---|---|---|
| | Concentration of | Yield | Contamination | Contamination |
| | DNA (ng/μL) | (%) | Level (260/280) | Level (260/230) |
| Cycle 1 | 3.5 | 60 | 1.86 | 2.41 |
| Cycle 2 | 4.1 | 70 | 1.86 | 2.39 |
| Cycle 3 | 4.2 | 72 | 1.86 | 2.40 |

As can be seen from the above three experiment results, as the cycle is repeated within each experiment, the concentration and yield of DNA are improved. This signifies that, as the eluant repeatedly passes through the filter unit 200, a DNA solution of a high concentration may be obtained at a high yield.

According to the illustrated embodiments of the microfluidic apparatus and the separation method according to the invention, DNA may be separated at a high concentration from a sample solution by using a small amount of eluant. That is, DNA of a considerable concentration may be obtained without a separate concentration process after purification.

Thus, the invention may be widely used in the fields of amplification and signal generation, such as a microarray needing high concentration DNA or a polymerase chain reaction (“PCR”) highly needing DNA concentration. Also, the invention may be used in a variety of molecular biological projects using high concentration DNA, such as molecular cloning, gene library generation, or DNA sequencing analysis. Also, a reaction efficiency in a restriction endonuclease reaction, a ligation reaction, an extension reaction, or the PCR, which are performed after the DNA extraction and purification process, may be improved.

According to the embodiment illustrated in FIG. 7, the eluant passing through the filter unit 200 is accommodated in the target substance accommodation chamber 310. In the state in which the valve 315 is closed, the eluant is transferred from the target substance accommodation chamber 310 to the eluant chamber 120. When the transfer is completed, the valve 125 is open and the eluant is supplied to the filter unit 200. Thus, a number of the repeated cycles of the separation process may be identified. In exemplary embodiments, to identify the exact number of cycles in the embodiments illustrated in FIGS. 1, 5, and 6, the separation time may be adjusted by appropriately setting the operation time of the resupply pump 520.

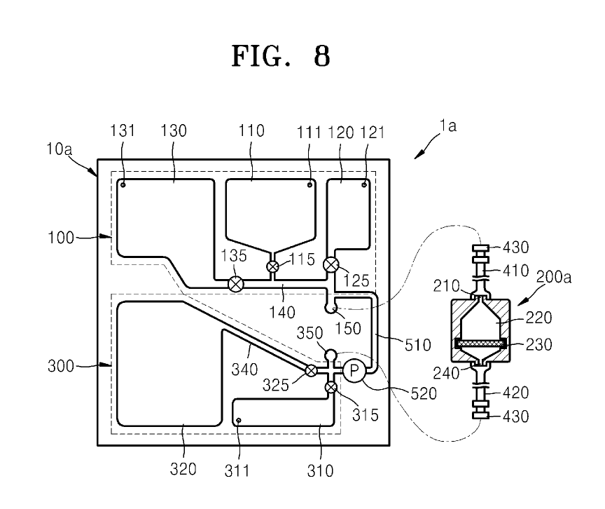

FIG. 8 is a plan view schematically illustrating another exemplary embodiment of a structure of a microfluidic apparatus 1a according to the invention. Referring to FIG. 8, a filter unit 200a is provided as a separate member from a remaining portion of the microfluidic apparatus 1a, such as by being separated from a platform 10a of the microfluidic apparatus 1a.

The filter unit 200a is fluidly connected to the supply channel 140 and the exhaust channel 340, respectively, via first and second connection members 410 and 420, which are of a tube type. A whole of the first and second connection members 410 and 420 are physically separate from the remaining portion of the microfluidic apparatus 1a, such that the first and second connection members 410 and 420 are the only connection of the filter unit 200a to the remaining portion of the microfluidic apparatus 1a.

First and second connection ports 150 and 350 included in the remaining portion of the microfluidic apparatus 1a, are respectively provided at the supply channel 140 and the exhaust channel 340. A first end portion of the first connection member 410 is connected to the entrance 210 of the filter unit 200a, and a second end portion of the first connection member 410 is connected to the first connection port 150 of the remaining portion of the microfluidic apparatus 1a. A first end portion of the second connection member 420 is connected to the exit 240 of the filter unit 200a, and a second end portion of the second connection member 420 is connected to the second connection port 350 of the remaining portion of the microfluidic apparatus 1a.

A sealing member 430 configured to prevent leakage of a fluid, is inserted at each of the second end portion of the first connection member 410 and the second end portion of the second connection member 420. Alternatively, the sealing member 430 configured to prevent leakage of a fluid may be inserted in each of the first and second connection ports 150 and 350. The first and second connection members 410 and 420 may be, for example, a flexible tube.

When the amount of a sample solution is relatively large, a large amount of the microparticles and the eluant are used. Accordingly, the size of the filter unit 200a increases so that it may be difficult to accommodate the filter unit 200a that is large in the platform 10a of the microfluidic apparatus 1a having a limited size. Also, filters formed of a variety of materials may be employed as the filter 230, according to the type of the target substance subject to purification. According to the microfluidic apparatus 1a of FIG. 8, the filter unit 200a having a size suitable for the purpose of use, and including the appropriate filter 230 may be used by being connected to the platform 10a.

Although the filter unit 200 or 200a of a sieve type is used in the above-described embodiments, the invention is not limited thereto. In one exemplary embodiment, the microparticles may be magnetic particles having magnetism, and the filter unit 200 or 200a may have a structure to filter microparticles using the magnetism. A magnet may be provided in the filter units 200 and 200a of the illustrated embodiments.

Although the exemplary embodiment of separating DNA only is described in the above-illustrated embodiments, the invention is not limited thereto. The microfluidic apparatus according to the above-illustrated embodiments may be used for purifying a variety of target substances using the eluant, the washing solution, and the microparticles having appropriate probes according to the type of the target substance subjection to the purification.

It should be understood that the exemplary embodiments described herein should be considered in a descriptive sense only and not for purposes of limitation. Descriptions of features or aspects within each embodiment should typically be considered as available for other similar features or aspects in other embodiments.

What is claimed is:

1. A microfluidic apparatus comprising:
a filter unit comprising an entrance, a separation portion in which eluant contacts microparticles to separate a target substance bound to surfaces of the microparticles from the microparticles, an exit, and a filter blocking leakage of the microparticles through the exit;
a fluid supply portion selectively supplying the eluant and a sample solution including the microparticles including the surfaces to which the target substance is bound, to the filter unit; and
a resupply unit supplying the eluant passed through the filter unit back to the filter unit.

2. The microfluidic apparatus of claim 1, wherein the resupply unit comprises:
a resupply path connecting the exit and the entrance of the filter unit to each other; and
a resupply pump affecting a transfer of the eluant along the resupply path and to the entrance of the filter unit.

3. The microfluidic apparatus of claim 1, wherein
the fluid supply portion comprises an eluant chamber accommodating the eluant, and
the resupply unit comprises a resupply path connecting the exit and the entrance of the filter unit to each other, and a resupply pump affecting a transfer of the eluant along the resupply path and to the entrance of the filter unit.

4. The microfluidic apparatus of claim 3, wherein the eluant chamber and the resupply pump are located within the resupply path.

5. The microfluidic apparatus of claim 4, further comprising a fluid accommodation portion accommodating the eluant exhausted through the exit of the filter unit and including a target substance accommodation chamber accommodating the eluant including the target substance separated from the microparticles;
wherein the eluant chamber, the target substance accommodation chamber and the resupply pump are located within the resupply path.

6. The microfluidic apparatus of claim 1, further comprising a fluid accommodation portion accommodating the eluant exhausted through the exit of the filter unit.

7. The microfluidic apparatus of claim 6, wherein the fluid accommodation portion comprises:
a target substance accommodation chamber accommodating the eluant including the target substance separated from the microparticles; and
an accommodation valve selectively connecting the target substance accommodation chamber and the exit of the filter unit.

8. The microfluidic apparatus of claim 7, wherein
the resupply unit comprises a resupply path connecting the exit and the entrance of the filter unit to each other, and a resupply pump affecting a transfer of the eluant along the resupply path and to the entrance of the filter unit; and
the target substance accommodation chamber and the resupply pump are located within the resupply path.

9. The microfluidic apparatus of claim 7, wherein the resupply unit comprises:
a resupply path connecting the target substance accommodation chamber and the entrance of the filter unit; and
a resupply pump affecting a transfer of the eluant along the resupply path and to the entrance of the filter unit.

10. The microfluidic apparatus of claim 7, wherein
the fluid supply portion comprises an eluant chamber accommodating the eluant, and
the resupply unit comprises a resupply path connecting the target substance accommodation chamber and the eluant chamber, and a resupply pump affecting a transfer of the eluant along the resupply path and to the entrance of the filter unit.

11. The microfluidic apparatus of claim 2, wherein only the resupply pump is located within the resupply path.

12. The microfluidic apparatus of claim 6, further comprising a platform on which the fluid supply portion, the fluid accommodation portion, and the resupply unit are disposed,
wherein the filter unit is physically separated from the platform, and the entrance and the exit of the filter unit are fluidly connected to the fluid supply portion, the fluid accommodation portion, and the resupply unit via first and second connection members, respectively.

13. A method of purifying a target substance, the method comprising:
packing microparticles including surfaces to which the target substance is bound, in a filter unit;
separating the target substance from the microparticles by supplying eluant to the filter unit; and
resupplying the eluant including the target substance exhausted from the filter unit, at least one time to the filter unit.
